(19) United States
(12) Patent Application Publication
(10) Pub. No.: US 2007/0059197 A1
(43) Pub. Date: Mar. 15, 2007

(54) Method of decontaminating, disinfecting or sterilizing of an article by means of an electrolytic system or by reactive oxygen species

(76) Inventors: Claus Jacob, St. Ingbert (DE); Fiona Fry, Devon (GB); Andrea Holme, Singapore (SG)

Correspondence Address: NIXON & VANDERHYE, PC, 901 NORTH GLEBE ROAD, 11TH FLOOR, ARLINGTON, VA, 22203, US

(21) Appl. No.: 10/576,111

(22) PCT Filed: Oct. 14, 2004
(86) PCT No.: PCT/GB04/04367

§ 371 Date: Aug. 16, 2006

(30) Foreign Application Priority Data
Oct. 14, 2003 (GB) 0324058.7

(51) Int. Cl. A61L 2/00; B01J 19/08

(52) U.S. Cl. 422/23; 422/186.04

(57) ABSTRACT

A method of decontaminating, disinfecting or sterilizing an article which method comprises placing the article in an electrolytic system with an oxygenated electrolyte (4′) and applying a potential difference to the electrolytic system. The method is particularly suitable for the sterilization of stainless steel surgical instruments (3′) wherein the instrument comprises the cathode in the electrolytic system.

The present invention relates to a method of and apparatus for decontaminating, disinfecting or sterilizing articles in particular by use of an electrolytic system with an oxygenated electrolyte. The method is particularly suitable for the sterilization of medical devices such as surgical instruments.

Decontamination of an instrument represents a combination of processes used to render the instrument safe for re-use. Although applicable to a variety of fields of industry, decontamination is of particular importance in the field of healthcare.

In general, the steps involved in a process of decontamination of a medical instrument are cleaning, disinfection and sterilization. Cleaning is a necessary first step in the decontamination, since it removes organic matter, which, in addition to posing a health risk in its own right, can inhibit the later disinfection and sterilization steps. The next step in the process is disinfection, which aims to remove or destroy pathogenic microorganisms, with the exception of bacterial spores. Disinfection is typically achieved through either automated washing and heating processes or by the application of chemicals. There are a number of chemical disinfectants which are commonly used which include general purpose detergents, such as household detergents (e.g. dishwashing liquid), chlorine preparations, (e.g. sodium hypochlorite) and alcohol preparations (e.g. 70% isopropyl alcohol). However, a number of these preparations, in particular the chlorine-containing disinfectants, can cause corrosion of surgical instruments. Disinfection can also be achieved using washer/disinfectors, which use a combination of automated cleaning and heat to remove microorganisms. The final step in the decontamination is sterilization which is aimed at the, ideally complete, removal of, ideally all, organisms from the medical instrument, including bacterial spores. Sterilization is generally performed using steam and requires contact between all surfaces of the instrument to be sterilized and steam at a specified pressure for a specified period of time. However, sterilization can also cause corrosion of a surgical instrument, particularly if tap water is used in the steam sterilizer. Of course, it is to be understood that disinfection and sterilization are not only performed within a process of decontamination and may be processes performed in their own right.

Decontamination of medical equipment is crucial to the minimisation of the risk of hospital acquired infections (HAI) for example MRSA. A further example of such infections are transmissible spongiform encephalopathies (TSEs) or “prion diseases” which are neurodegenerative disorders of the central nervous system that lead to motor dysfunction, dementia and eventually death. An example of such a TSE is Creutzfeldt Jakob Disease (CJD). There is no known risk of TSE transmission through normal clinical or social contact with an infected person, however, patients have been infected when undergoing neurosurgery, comeal and dura matter transplantation as well as injection of human growth hormone (Decontamination of Surgical Instruments and Other Medical Devices—Report of a Scottish Executive Health Department Working Group February 2001).

Bovine spongiform encephalopathy (BSE) and variant CJD (vCJD) are also examples of TSEs. The agent that causes BSE and vCJD has not yet been verified but it is known that an improperly folded form of a membrane prion protein (PrP) is involved. This disease-specific form of PrP, termed PrP^{Sc}, has been found in the tonsils, spleen and lymph nodes and CNS of patients who have died as a result of contracting vCJD.

Prion proteins are an issue in medical decontamination because they have extreme resistance to conventional sterilisation techniques and a tendency to bind to the surfaces of metal and plastic without loss of infectivity (PNAS Colloquium; 2002; 99, suppl. 4; 16378-16383). Sterilisation of PrP^{Sc} by heat currently involves exposure of the protein to steam at 134° C. for 18 minutes in a porous load autoclave. However, it has been suggested that longer exposure times may be required, and even so, complete inactivation may not be possible by this method. In a case that highlights the infectivity of prion proteins, an electrode that had previously been inserted into the cortex of an undiagnosed CJD-infected patient was subjected to a decontamination procedure that involved treatment with benzene, 70% ethanol and formaldehyde vapour (J. Neurol. Neurosurg. Psychiatry; 1994; 57, 757-758). This same electrode was subsequently used on two patients, both of whom developed CJD within two years of treatment. The electrode had been cleaned as above after each use. The electrode, recognised as the culprit, was then inserted into the brain of a chimpanzee, where it also caused spongiform encephalopathy. This investigation showed that the electrode continued to possess infectious prion proteins even after several years and numerous attempts at sterilization. A method of sterilization which was able to remove prion proteins from the surface of a medical instrument would therefore represent an significant improvement over the prior art.

It is an object of the present invention to provide an improved method of decontamination, disinfection or sterilization.

According to a first aspect, the present invention provides a method of decontaminating, disinfecting or sterilizing an article which method comprises placing the article in an electrolytic system with an oxygenated electrolyte and applying a potential difference to the electrolytic system. It is believed that contaminants, for example contaminating biological matter, can be effectively removed from an article using the method of the present invention. It is also believed that contaminants, for example contaminating biological matter, on the surface of an article can be destroyed using the method of the present invention. It is further believed that the method serves to degrade, or otherwise render ineffective, contaminants present on the surface or contaminants that are present in the electrolyte (for example contaminants that become removed from the surface during the method). It is further believed that the method of the present invention can inhibit re-adhesion of contaminants to the article. The contaminants that can be removed using the present invention are believed to include amino acids, proteins, bacteria, mammalian cells and prion proteins. The method of the present invention is particularly suitable as a method of sterilizing an article.

The method of the present invention is suitable for the decontamination, disinfection or sterilization of any article. However, it is particularly suitable for the decontamination, disinfection or sterilization of medical instruments, for example surgical instruments or dental instruments. The medical instruments are typically medical conductive instruments such as medical instruments which comprise metal. Preferably, the medical instrument is a surgical instrument. Examples of such surgical instruments are scalpels, electrodes and surgical screws. Further examples of articles for use in the method of the present invention are contact lenses.

The electrolyte that can be used in the electrolytic system of the present invention can be any electrolyte within which oxygen may be reduced. The skilled person would be aware of electrolytes which are suitable for this purpose. Typical electrolytes are aqueous solutions of ionic salts. Examples of such electrolytes are aqueous solutions of potassium hydroxide, sodium phosphate, sodium chloride, sodium sulphate, sodium hydrogen carbonate, orthoborate or citrate. Preferred electrolytes are environmentally friendly electrolytes such as aqueous solutions of sodium phosphate, sodium chloride, sodium sulphate, sodium hydrogen carbonate, orthoborate or citrate or mixtures thereof. A particularly preferred electrolyte is an aqueous solution of sodium hydrogen carbonate.

The pH of the electrolyte can affect the reduction of oxygen in the electrolytic system. For example, it is known that oxygen can undergo electro-reduction to produce the superoxide ion. This superoxide ion is a basic species and its stability decreases with pH. It is therefore preferred that the electrolyte is either neutral or alkaline. A typical range for the pH for the electrolyte is from 7 to 11. Due to environmental considerations, it is preferred that the pH of the electrolyte is substantially pH 7.

The present inventors have found that the presence of impurities in the electrolyte can reduce the rate of oxygen reduction. In light of this, when using aqueous electrolytes, it is preferable to use deionised water to prepare the electrolytic solution.

However, additives in the electrolyte can, in some cases, enhance the reduction of oxygen in the electrolytic system. An example of such an additive is detergent and the present inventors have found that a detergent concentration of 0.1 ml per ml of electrolyte or greater can enhance the increase the rate of reduction of oxygen. The present invention thus also relates to electrolytes which comprise additives wherein said additives enhance the reduction of oxygen in the electrolytic system

Oxygenation of the electrolyte is achieved by any method suitable for introducing oxygen (preferably molecular oxygen) into the electrolytic solution. Typical methods include bubbling an oxygen-containing gas (preferably a molecular oxygen-containing gas) through the electrolyte. Such a method is typically performed until the electrolyte is saturated with oxygen or the oxygen containing gas, although complete saturation of the electrolyte may not be necessary. It is envisaged that any oxygen-containing gas could be used for this purpose, however, it would typically be air. A precursor, which provides oxygen during the conditions of the reaction, could also be used to oxygenate the electrolyte.

Typically, the oxygen content in the electrolyte, in terms of the saturation level of oxygen, is from 1 to 100%. Preferably, the oxygen content is from 25 to 100%. More preferably, the oxygen content is from 50 to 100%. Particularly preferably, the oxygen content is 75 to 100%.

In a preferred embodiment, the oxygen content in the electrolyte, in terms of the saturation level of gas, is from 1 to 100%. Preferably, the oxygen content is from 15 to 100%. More preferably, the oxygen content is from 50 to 100%. Particularly preferably, the oxygen content is 75 to 100%.

The electrolyte is typically oxygenated before a voltage is applied to the electrolytic cell. However, oxygen can be introduced into the electrolyte during electrolysis.

The potential difference applied to the electrolytic system is typically a potential sufficient to reduce the oxygen in the oxygenated electrolyte. Generally, the potential difference applied to the system is less than that sufficient to reduce water to hydrogen gas. The potential difference which is sufficient to reduce the oxygen in the oxygenated electrolyte will be dependent upon numerous factors including the nature of the electrolyte, the pH of the electrolyte and the cathode. However, in general the range of the potential difference will be from −1.50 to +0.25 V compared with or versus the standard silver electrode (SSE). A preferred range for the potential difference is −1.25 to −0.5 V. A more preferred range is −1.0 to −0.8 V. As an example, for a polished stainless steel cathode in an alkaline solution the range of the potential difference for oxygen reduction is −0.2 to −0.9 V.

The method of the present invention can be performed at any temperature generally used for electrolytic systems. An example of a suitable temperature range is 10 to 75° C. A preferred temperature range is 15 to 50° C. It is envisaged that the method will generally be performed at room temperature.

The time for which the potential difference is applied to the electrolytic system will be dependent upon many factors including the nature of the electrolytic system, the voltage applied and the oxygen content of the electrolyte. However, it is envisaged that the potential difference will be applied for a time of up to about 36 hours. A preferred time limit is from 6 to 30 hours and a particularly preferred time limit is from 8 to 24 hours. In a preferred embodiment, the time limit is typically from 0.5 to 4 hours, preferably from 0.75 to 2 hours and more preferably is around 1 hour.

The cathode of the electrolytic system may be made of any suitable electrically conducting material that will allow oxygen to be reduced on its surface. Examples of such materials are stainless steel and related alloys. Further examples of such materials are conductive polymers.

The method of the present invention can effectively degrade biological material, such as amino acids and proteins, present in the electrolyte of the electrolytic system. Thus, in one embodiment, the present invention relates to a method, as described above, for decontaminating, disinfecting or sterilizing an article wherein the article is placed in the electrolyte.

The method of the present invention has been found to be particularly effective for degrading biological material, such as proteins, bacteria and mammalian cells, present on the surface of the cathode. Thus, in a preferred embodiment, the present invention provides a method, as described above, of decontaminating, disinfecting or sterilizing an article wherein the article comprises the cathode of the electrolytic system. Investigations have shown that the method of the present invention preserves the integrity of the cathode and does not therefore damage the article to be decontaminated, disinfected or sterilized. The method is therefore advantageous over a method which comprises direct anodic oxidation of the contaminants, since such a method is frequently associated with damage to the electrode. In this embodiment, the article can comprise any material provided that it can act as a cathode. Suitable materials include metals such as steel or brass, but stainless steel is preferred.

Reactive oxygen species may be produced by the method above using an electrolytic system and an oxygenated electrolyte. Another aspect of the present invention provides the use of a reactive oxygen species in the decontamination, disinfection or sterilization of an article. In particular, the present invention provides the use of a reactive oxygen species in the sterilization of an article. Preferred reactive oxygen species are one or more of the superoxide ion (O₂⁻⁰), the hydroxyl radical (OH⁰) and hydrogen peroxide (H₂O₂). It is further preferred when one of the reactive oxygen species is the hydroxyl radical or the superoxide ion. Particularly preferred is when one of the reactive oxygen species is the superoxide ion. It is most preferred when the reactive oxygen species is the superoxide ion. In one embodiment, it is most preferred when the reactive oxygen species is the hydroxyl radical.

A further aspect of the present invention provides a decontamination, disinfection or sterilization apparatus which comprises an electrolytic system with an oxygenated electrolyte. The present invention also provides a decontamination, disinfection or sterilization apparatus which comprises an electrolytic system which, in use, comprises an oxygenated electrolyte. The present invention also provides such an apparatus which comprises a container with a detachable lid. In such an apparatus part of the circuit wire of the electrolytic system can be contained in the lid such that the circuit is completed when the lid is placed on the container and broken when it is removed. The present invention further provides such an apparatus wherein the cathode of the electrolytic system is the article to be sterilized. The present invention additionally provides such an apparatus which comprises means for passing current through the article to be decontaminated, disinfected or sterilized such that, in use, the article acts as the cathode of the electrolytic system. The present invention further provides an apparatus wherein the article to be decontaminated, disinfected or sterilized is held between the anode and the cathode of the electrolytic system. In such an apparatus the article can be held in a holder attached to the lid. Further, the holder can be made of made of a non-conductive material, such as non-conductive plastic mesh. The present invention also provides a decontamination, disinfection or sterilization apparatus for performing a method of decontamination, disinfection or sterilization as herein described.

The invention will be further described by way of example, with reference to the accompanying drawings, in which

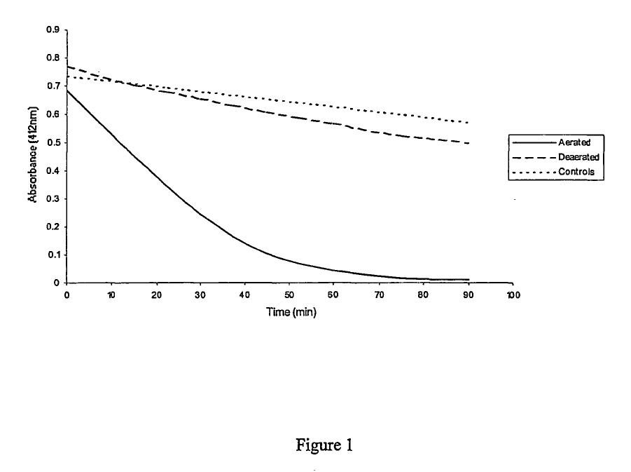

FIG. 1 shows DTNB assays for glutathione for a control, an aerated and a deaerated electrolyte;

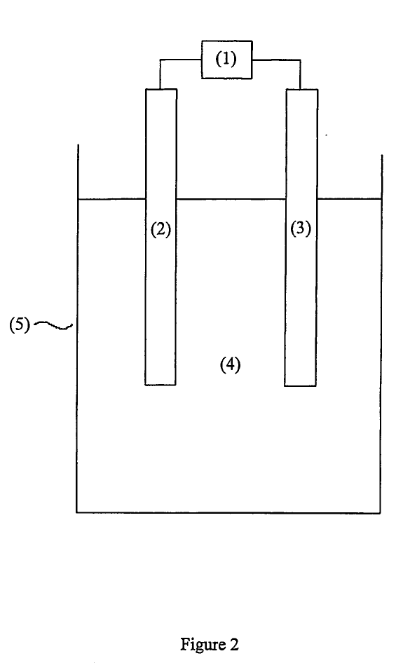

FIG. 2 schematically illustrates an electrolytic system in accordance with one embodiment of the invention;

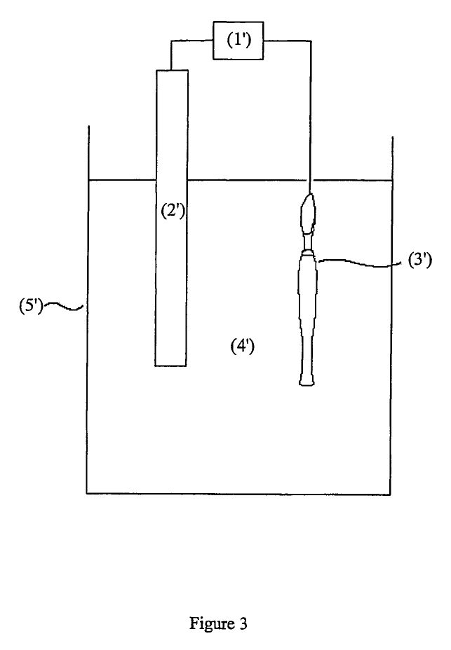

FIG. 3 schematically illustrates an electrolytic system in accordance with an embodiment of the invention wherein the article to be decontaminated, disinfected or sterilized forms the cathode of the electrolytic system; and

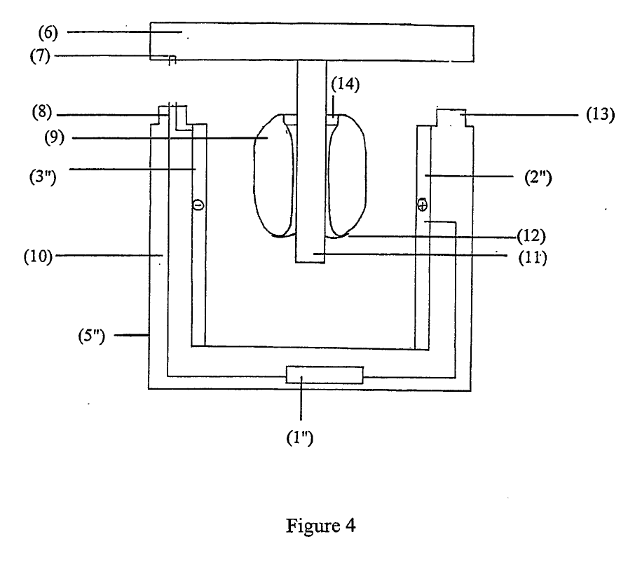

FIG. 4 schematically illustrates an electrolytic system in accordance with an embodiment of the invention wherein the article to be decontaminated, disinfected or sterilized is held in the between the anode and the cathode of the electrolytic system.

FIG. 2 schematically shows an electrolytic system which comprises: a power source 1 for applying a potential difference to the electrolytic system; an anode 2; a cathode 3; an oxygenated electrolyte 4; and a vessel 5.

FIG. 3 schematically shows an electrolytic system which comprises: a power source 1′ for applying a potential difference to the electrolytic system; an anode 2′; an article to be decontaminated, disinfected or sterilized 3′; an oxygenated electrolyte 4′; and a vessel 5′.

FIG. 4 schematically shows an electrolytic system which comprises: a lid 6; a circuit wire 7; a contact wire 8; a device for holding the article to be decontaminated, disinfected or sterilized 9; a cathode 3″; a circuit wire 10; a vessel 5″ which can be made from a non-conductive material such as a non-conductive plastic; a power source 1″; a central shaft 11 which is, for example, made of a non-conductive material such as a plastic mesh; a retainer clip 12; an anode 2″; a connection between the container and the lid 13 such a screw threaded or push-fit connection; and a hinge 14.

The embodiment shown in FIG. 4 can be used to decontaminate, disinfect or sterilize any article which can be placed in the basket, such as medical instruments. However, this embodiment is particularly suitable for the cleaning of contact lenses.

The anode 2 may be made of any suitable conducting material. Examples of suitable materials include any conductive material that does not corrode under these conditions. Particular examples are Noble metals, for example platinum, and carbon materials such as graphite.

In the electro-reduction of oxygen in an electrolytic system, oxygen is typically reduced at the cathode 3. However, the reduced oxygen species produced at the cathode 3 can be free in the electrolyte 4 and can also undergo reactions in the electrolyte 4 to produce further oxygen electro-reduction species i.e. reactive oxidizing oxygen species. The method of the present invention can therefore be performed by placing the article to be sterilized into the electrolyte 4 of the electrolytic system. However, in relation to the sterilization of a conductive or other metallic article, since the reduction of oxygen can occur at the surface of the cathode 3, it is a preferred embodiment of the present invention that the instrument to be sterilized is used as the cathode 3. It is to be understood that all or part of the conductive or other metallic article can comprise a conductive or metallic material. Furthermore, all or part of the surface of the conductive or other metallic article can comprise a conductive or metallic material. This embodiment is particularly suitable for stainless steel articles, for example stainless steel surgical instruments.

Surgical instruments typically comprise stainless steel. There are three types of stainless steel used in the manufacture of surgical instruments, martensitic, austenitic and ferritic. Martensitic stainless steel consists of iron (72-89.3%), chromium (10.5-18%) and carbon (0.2-1%). Martensitic stainless steel is moderately resistant to corrosion, and is used to make istruents that are required to have cutting edges, and to be strong and resistant to wear. Examples of martensitic stainless steel surgical instruments include surgical needle holders and dental scalers.

Austenitic stainless steel consists of iron (62-78%), chromium (16-26%) and nickel (6-12%). This type of stainless steel is used to manufacture medical instruments that require good corrosion resistance and moderate strength. Examples of austenitic stainless steel medical instruments include hypodermic needles and dental impression trays. Ferritic stainless steel consists of iron (approximately 82-87%), chromium (12.5% or 17%) and nickel (typically <1%). This type of stainless steel finds relatively little use in the manufacture of medical instruments. Examples of ferritic stainless steel medical instruments include medical devices with screw heads and solid handles for instruments.

In this embodiment of the present invention, when a potential difference is applied to electrolytic system, the oxygen in the oxygenated electrolyte can undergo a series of reactions which produce reactive oxygen species. Examples of such reactive oxygen species are the superoxide ion (O₂⁻⁰), the hydroxy radical (OH⁰) and hydrogen peroxide (H₂O₂). The efficacy of the method of decontamination, disinfection or sterilization described herein is presently ascribed to the reactive oxygen species which are produced under the conditions of the reaction.

### EXAMPLES

Materials and Methods

Surgical Instrument Electrode

A 2-inch section of a surgical tissue clamp was cut and used as the surgical instrument electrode.

Electrochemistry

The electrochemistry machine used was a BAS-50W Voltammetric analyser.

Chemicals

All chemicals were of the highest purity available and were acquired from Sigma-Aldrich, unless otherwise stated.

Mass Spectrometry

All mass spectrometry was performed on a Micromass LC analyser. All mass spectrometry was done in electrospray positive mode, with a cone voltage of 30 V.

Electrolysis

Cyclic Voltammetry. All Cyclic Voltammetry was performed by the following method. All potentials are quoted against the Ag/AgCl reference electrode (standard silver electrode, SSE), unless otherwise stated.

A 20 ml aliquot of electrolyte was used in a standard electrochemical cell. The working electrode was polished with polishing alumina before each use. The auxiliary electrode was either a graphite rod (6 mm diameter), or a platinum coil. The reference electrode was Ag/AgCl. The initial scan direction was negative. A magnetic stirrer was used to stir the solution. For deaerated solutions, nitrogen was passed through the solution for one hour before electrolysis. After one hour, the nitrogen outlet tube was removed from the electrolyte, and placed over its surface. This maintained a blanket of nitrogen over the electrolyte surface throughout electrolysis.

Glutathione Degradation—Assessment by DTNB Assay

Bulk electrolysis was run for 90 minutes. A 100 μM sample was removed from the elecrolysis cell every 15 minutes. This sample was added to a cuvette containing phosphate buffer (pH 7) 100 mM and 100 μM 5.5′-dithiobis(2-nitrobenzoic acid) (DTNB) in deionised water. The absorbance was read at 412 nm. A control was left standing at room temperature for 90 minutes, and then 100 μl was added to the EDTA and DNTB solution and the absorbance was read at 412 nm.

Glutathione Degradation—Assessment by Mass Spectrometry

10 μl of 100 mM glutathione stock solution was added to a bulk electrolysis cell and run as described for 90 minutes. The initial concentration of glutathione in the electrolysis cell was 50 μM. A sample was then removed (20 μl) and diluted to 5 ml with a solution of 49.5% acetonitrile +49.5% deionised water +1% formic acid. The final GSH concentration was 200 nM. A GSH control was also prepared, so that the final GSH concentration was 200 nM. These solutions were run in the electrospray mass spectrometer.

Assessment of Myoglobin Degradation by Mass Spectrometry

Myoglobin stock solutions were made to 10 mg/ml concentration and de-salted on a PD-10 column flushed with deionised water.

1 ml of a 10 mg/ml myoglobin stock solution was added to 19 ml electrolyte. Bulk electrolysis was run as described for 8 hours. A sample was removed every hour. One part protein solution was added to two parts of a 49.5% acetonitrile +49.5% deionised water +1% formic acid solution. A control was allowed to sit at room temperature. The control was made up of 1 ml stock myoglobin solution and 19 ml NaHCO₃ pH 7, 50 mM. The solutions were run in the electrospray mass spectrometer. The control solution and all protein samples were stored at −20° C. until use.

Assessment of Myoglobin Degradation by SDS-PAGE

A 15% acrylamide gel was prepared from the following:

Resolving Gel:

4.24 ml bisacrylamide

3.995 ml Resolving buffer mix

1 ml ammonium persulphate

10 μl TEMED

4.769 ml Water

Stacking Gel:

0.3034 ml bisacrylamide

2.03 ml Stacking buffer mix

0.3 ml ammonium persulphate

4 μl TEMED

0.669 ml water

The gels were run at 100 mV, stained with coomassie blue and then destained with standard SDS-PAGE gel destaining solution.

Bulk Electrolysis on the Surgical Instrument

All bulk electrolysis experiments were performed as follows. The working electrode was the surgical instrument. The magnetic stirrer was set to a rate of 700 rpm. The auxiliary electrode was a graphite rod. The fixed potential was −900 mV. 50 mM NaHCO₃, pH 7, electrolyte was used.

### Example 1

Glutathione Degradation

Assessment of Glutathione Degradation by the DTNB Assay

Glutathone (GSH) is a tripeptide consisting of cysteine, glycine and glutamate. 5,5′-Dithiobis(2-nitrobenzoic acid) (DTNB) reacts with SH groups to produce 5-mercapto-2-nitrobenzoic acid, which absorbs light at a wavelength of 412 nm. As GSH has a sulfhydryl group, DTNB can be used to assay the amount of GSH present in a sample. In this assay, glutathione was added to the electrolyte so that the final GSH concentration was 50 μM. The surgical instrument was used as the working electrode, and was held at a constant potential of −900 mV for 90 minutes. A sample was removed from the electrochemical cell at 15-minute intervals and used in the DTNB assay. This was done in triplicate for both aerated and deaerated solution. Controls containing glutathione in the electrolyte were left to stand at room temperature for 1 hour. FIG. 1 compares the results of the DTNB assays for aerated and deaerated solutions.

The results of the DTNB assay show a clear difference between the aerated and deaerated solutions. The results of the assay for the aerated solution indicate that the surgical instrument working electrode degrades glutathione in NaHCO₃ pH 7. The absorbance decreases linearly until after about 40 minutes, when it begins to plateau. This is consistent with increasing production of reactive oxygen species by the reduction of oxygen in the electrolyte, until a maximal rate is achieved, limited by mass transport of both O₂ and GSH to the working electrode and the O₂ depletion of the solution. In the deaerated solution, some degradation of glutathione seems to occur, but at a rate only slightly faster than the control. The graph of absorbance against time for the deaerarated solution closely follows the graph for the control. This suggests that either the degassing was incomplete, and some oxygen remained in the electrolyte, or that GSH is oxidised to some degree by a process not involving oxygen at the auxiliary electrode.

Assessment of Glutathione Degradation by Mass Spectrometry

Glutathione (GSH) was added to 50 mM NaHCO_{3(aq)} pH 7 electrolyte so that the final GSH concentration was 50 μM. The surgical instrument was used as the working electrode, and was held at a constant potential of −900 mV for 90 minutes. At 90 minutes, a sample was removed from the electrochemical cell and run in an Electrospray (ES) mass spectrometer. A control containing glutathione in the electrolyte was left to stand at room temperature for 90 minutes.

The molecular weight of glutathione is 307 and the control solution showed a peak at approximately m/z 308 due to protonated glutathione. This peak for glutathione is presenting the mass spectrum for the control solution, but absent from the mass spectrum for the aerated solution. This indicates that glutathione is degraded by oxygen reduction at −900 mV on the surgical instrument electrode in aqueous NaHCO₃ pH 7 electrolyte. A peak also appeared at approximately 615 due to GSSG, the oxidised form of glutathione, which is present as a small proportion of total peptide.

### Example 2

Myoglobin Degradation

Assessment of Myoglobin Degradation by Mass Spectrometry

1 ml of a 10 mg/ml myoglobin stock solution was added to 19 ml electrolyte. Bulk electrolysis was run for 8 hours with the surgical instrument as the working electrode, at a potential of −900 mV. A sample was removed every hour and stored in the fridge. This was done for both aerated and deaerated solution. A control containing myoglobin in NaHCO₃ (pH7) was allowed to sit at room temperature for 8 hours. The control was made up to the same concentration as the sample. Following electrolysis, the solutions were run in an Electrospray-mass spectrometer.

In the mass spectrum of the stock solution, peaks appeared between m/z 700 and m/z 1600 corresponding to differently protonated forms of myoglobin. The molecular mass of horse heart myoglobin is approximately 17,000 g/mol. The peaks at m/z 707, 734, 771, 808, 849, 893, 943, 998, 1060, 1131, 1138, 1219, 1319 and 1413 correspond to myoglobin protonated between 24-12 times, respectively.

There was a decrease in the peak heights corresponding to myoglobin in the control, compared to the stock solution. Additional peaks were also observed between m/z 600 and m/z 94 in the control mass spectrum, which were not present in the mass spectrum for the stock solution. These peaks could correspond to the breakdown products of myoglobin in the control. In particular, there were relatively large peaks at m/z 91 and 227 which were absent from the control spectrum. It is suggested that these differences in the mass spectra for the stock and control solutions were due to normal degradation seen when freeze-thawing proteins.

The mass spectra for the deaerated solution indicated that even after eight hours of bulk electrolysis, there was only a small relative difference compared to the control. The mass spectra for the aerated solution, on the other hand, clearly indicated a large relative difference between the samples removed from bulk electrolysis and the control. After eight hours of electrolysis at −900 mV, the myoglobin peak had almost completely disappeared. Comparison of the spectra for the 8-hour samples from the aerated and deaerated solutions indicates that such extensive degradation occurs only in the aerated solution.

Assessment of Myoglobin Degradation by SDS-PAGE

The same samples used for the mass spectrometric analysis were also analysed by polyacrylamide gel electrophoresis (PAGE).

The size markers used were broad range size markers, consisting of nine peptides with molecular weights of 225, 150, 100, 75, 50, 35, 25, 15 and 10 kDa. There was a clear difference between the gels for the deaerated and aerated solutions. The gel for the deaerated solution indicated that after incubation for eight hours in the electrolysis cell, there is little observable difference in the intensity of the myoglobin bands compared to the control. In the gel for the aerated solution, there is a clear decrease in intensity after five hours compared to the control and to the equivalent lane in the gel for the deaerated solution. The intensity of the bands decreased even further for the 6, 7 and 8-hour samples. After electrolysis for 8 hours in aerated electrolyte, the sample gives only a very faint band corresponding to intact myoglobin, compared to the control.

Experiment 3

Dialysis Tubing Experiments

Albumin protein was added into dialysis tubing (cut of range of 12-14,000 Da) and the tube attached to one of the electrodes. The protein was thereby kept in close contact to one of the electrodes, either the surgical instrument cathode or the graphite rod anode. Electrolysis was then performed. The results of this experiment were that protein was degraded at the surgical instrument cathode but not at the graphite anode. This confirmed that the process of decontamination is cathodic which, together with the aerobic/nitrogen experiments, supports the assertion that the decontamination proceeds via a reductive, oxygen radical process. The experiment also showed that contamination close to the instrument surface can be effectively destroyed. Most instruments contain contaminants adsorbed on the surface, and this experiment is a close reflection of the real life situation.

Experiment 4

Bacteria and Mammalian Cells

Since the method proved effective against proteins, its effects against bacteria were also studied. Bacteria (E. coli) were allowed to adsorb directly onto the metal by placing the metal in the bacteria solution for 2 hours before air drying. Upon exposure of the adsorbed bacteria to the system for 24 hours, there was a significant decrease in both the number and size of the bacterial colony growth when the samples were applied to an Agar bacterial growth medium. In this experiment, the metal was swabbed under aseptic conditions using a plastic sterile loop and streaked out onto a freshly prepared LB agar plate and left to grow overnight at 37° C. Colonies were then counted as indicator of bacteria survival.

In order to study efficiency against mammalian cell contamination, the metal was coated with poly-D-lysine and Daudi cells were allowed to adhere to the metal for 24 hours. The choice of Daudi cells was due to them being of a blood cell type known as B cells. The cells were then exposed to the decontamination system for 24 hours before the metal was tested for contamination using the commercially available ninhydrin test method. Ninhydrin reacts with amino groups, developing a red colour. The positive and negative controls that came with the kit were therefore red and colourless, respectively. The metal which had been ‘decontaminated’ in the system according to the present invention did not show a red colour when stained with Ninhydrin while the positive control metal, which was treated in the same way apart from not having any current applied to the system and therefore was not decontaminated did show a red colour. This is a clear indication that the mammalian cells (and poly-D-lysine) were removed from the instrument surface.

1. A method of decontaminating, disinfecting or sterilizing an article which method comprises placing the article in an electrolytic system with an oxygenated electrolyte and applying a potential difference to the electrolytic system.

2. A method according to claim 1 wherein the method is a method of sterilizing an article.

3. A method according to claim 1 wherein the article is a medical instrument.

4. A method according to claim 1 the article is a surgical instrument.

5. A method according to claim 1 wherein the article comprises stainless steel.

6. A method according to claim 1 wherein the article comprises the cathode of the electrolytic system.

7. A method according to claim 1 wherein the electrolyte is an aqueous solution of sodium phosphate, sodium chloride, sodium sulphate, sodium hydrogen carbonate, orthoborate or citrate.

8. A method according to claim 7 wherein the electrolyte is an aqueous solution of sodium hydrogen carbonate.

9. A method according to claim 1 wherein the electrolyte is either neutral or alkaline.

10. A method according to claim 9 wherein the pH of the electrolyte is substantially 7.

11. A method according to claim 1 wherein the electrolyte is aerated prior to use.

12. A method according to claim 1 wherein the electrolyte is aerated during the electrolysis.

13. A method according to claim 1 wherein the oxygen content of the oxygenated electrolyte is from 1 to 100%.

14. A method according to claim 13 wherein the oxygen content of the oxygenated electrolyte is from 50 to 100%.

15. A method according to claim 1 wherein the potential difference applied to the electrolytic system is from −1.50 to 0.25 V compared with or versus the standard silver electrode (SSE).

16. A method according to claim 15 wherein the potential difference is from −1.25 to −0.5 V compared with or versus the standard silver electrode (SSE).

17. A method according to claim 1 wherein the potential difference is applied to the electrolytic system for up to 36 hours.

18. A method according to claim 17 wherein the potential is applied for 6 to 24 hours.

19. Use of a reactive oxygen species in the decontamination, disinfection or sterilization of an article.

20. Use according to claim 19 in the sterilization of an article.

21. Use according to claim 19 wherein the reactive oxygen species comprises one or more of the superoxide ion (O₂⁻⁰), the hydroxyl radical (OH⁰) and hydrogen peroxide(H₂O₂).

22. Use according to claim 21 wherein one of the reactive oxygen species is the hydroxyl radical or the superoxide ion.

23. A decontamination, disinfection or sterilization apparatus which comprises an electrolytic system with an oxygenated electrolyte.

24. (canceled)

25. An apparatus according to claim 23 which comprises a container with a detachable lid.

26. An apparatus according to claim 25 which comprises a container with a lid and wherein part of the circuit wire of the electrolytic system is in the lid such that the circuit is completed when the lid is placed on the container and broken when it is removed.

27. An apparatus according to claim 23 wherein the cathode of the electrolytic system is the article to be sterilized.

28. An apparatus according to claim 23 which comprises means for passing current through the article to be decontaminated, disinfected or sterilized such that, in use, the article acts as the cathode of the electrolytic system.

29. An apparatus according to claim 23 wherein the article to be decontaminated, disinfected or sterilized is held between the anode and the cathode of the electrolytic system.

30. An apparatus according to claim 29 wherein the article is held in a holder attached to the lid.

31. An apparatus according to claim 29 wherein the holder is made of non-conductive plastic mesh.

32. A decontamination, disinfection or sterilization apparatus for performing a method as described in claim 1.

33. A decontamination, disinfection or sterilization apparatus which comprises an electrolytic system which, in use, comprises an oxygenated electrolyte.

34. A method according to claim 1 wherein the article is a contact lens.
